(19) United States
(12) Patent Application Publication
(10) Pub. No.: US 2005/0153314 A1
(43) Pub. Date: Jul. 14, 2005

(54) Differential gene expression in intestinal polyps

(76) Inventors: David de Graaf, Cambridge, MA (US); Eric S. Lander, Cambridge, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/961,819

(22) Filed: Oct. 8, 2004

Related U.S. Application Data
(63) Continuation of application No. 09/916,179, filed on Jul. 25, 2001, now abandoned.
(60) Provisional application No. 60/220,579, filed on Jul. 25, 2000.

(51) Int. Cl. C12Q 1/68; G01N 33/574

(52) U.S. Cl. 435/6; 435/7.23

(57) ABSTRACT

The present invention features method for identifying an intestinal polyp according to the gen expression profile obtained from intestinal tissue. The present invention also features methods for identifying a compound that modulates intestinal polyp development, as well as oligonucleotide microarrays having genes involved in intestinal polyp development immobilized thereon.

### RELATED APPLICATION

This application is a continuation of U.S. application Ser. No. 09/916,179, filed on Jul. 25, 2001, which claims the benefit of U.S. Provisional Application No. 60/220,579, filed on Jul. 25, 2000, the entire teachings of which are incorporated herein by reference.

### BACKGROUND OF THE INVENTION

Intestinal polyps are a common type of intestinal disorder, and are found in many diseases, including Gardner syndrome, Peutz-Jeghers syndromes, familial juvenile polyposis, and familial adenomatous polyposis coli. Most polyps do not cause symptoms and are found incidentally during a regular cancer screening exam or in the investigation of gastrointestinal complaints, yet identification and removal of these polyps are crucial to preventing intestinal cancer.

Classification of biological samples from individuals is not an exact science. In many instances, accurate diagnosis and safe and effective treatment of a disorder depend on being able to discern biological distinctions among cell or tissue samples from a particular area of the body, such as intestinal polyp samples and normal intestinal samples. The classification of a sample from an individual into particular disease classes has often proven to be difficult, incorrect, or equivocal. Typically, using traditional methods such as histochemical analyses, immunophenotyping, and cytogenetic analyses, only one or two characteristics of the sample are analyzed to determine the sample's classification. Inaccurate results can lead to incorrect diagnoses and potentially ineffective or harmful treatment. Thus, a need exists for an accurate and efficient method for identifying polyps and differentiating between polyps and normal tissue.

### SUMMARY OF THE INVENTION

The present invention features methods of identifying an intestinal polyp, methods for identifying a compound that modulates intestinal polyp development, and oligonucleotide microarrays containing probes for genes involved in intestinal polyp formation.

In one aspect, the invention features a method of identifying an intestinal polyp, comprising obtaining a nucleic acid sample derived from intestinal tissue; and determining a gene expression profile from a gene expression product of at least one informative gene having increased expression in an intestinal polyp relative to a control. Increased expression of the informative gene in the sample is indicative of an intestinal polyp. In one embodiment, the intestinal polyp is an upper intestinal polyp or a colonic polyp. In another embodiment, the nucleic acid sample derived from intestinal tissue is derived from upper intestinal tissue or colonic tissue. In other embodiments, the gene expression product is DNA or mRNA. Preferably, when the gene expression product is DNA or mRNA, the gene expression profile is determined utilizing specific hybridization probes. For example, the gene expression profile may be determined utilizing oligonucleotide microarrays.

In another embodiment of the first aspect of the invention, the gene expression product is a polypeptide. Preferably, when the gene expression product is a polypeptide, the gene expression profile is determined utilizing antibodies.

In still another embodiment, the one or more informative genes is selected from the group consisting of apoptosis genes, cell cycle genes, tumor suppressor genes, cell adhesion genes, transcription related genes, and inflammation genes. In a preferred embodiment, the one or more informative genes is selected from the group consisting of the genes in FIGS. 1A-1U.

The invention also features a method of identifying an intestinal polyp, comprising obtaining a polypeptide sample derived from intestinal tissue; and determining a gene expression profile from a gene expression product of at least one informative gene having increased expression in an intestinal polyp relative to a control, where the gene expression product is a polypeptide. Increased expression of the gene expression product in the sample is indicative of an intestinal polyp. In one embodiment, the intestinal polyp is an upper intestinal polyp or a colonic polyp. In another embodiment, the polypeptide sample derived from intestinal tissue is derived from upper intestinal tissue or colonic tissue. In another embodiment, the gene expression profile is determined utilizing antibodies. In yet another embodiment, the one or more informative genes is selected from the group consisting of apoptosis genes, cell cycle genes, tumor suppressor genes, cell adhesion genes, transcription related genes, and inflammation genes. In a preferred embodiment, the one or more informative genes is selected from the group consisting of the genes in FIGS. 1A-1U.

In addition, the invention features a method of identifying an intestinal polyp, comprising obtaining a nucleic acid sample derived from intestinal tissue; and determining a gene expression profile from a gene expression product of at least one informative gene having decreased expression in an intestinal polyp relative to a control. Decreased expression of the gene in the sample is indicative of an intestinal polyp. In one embodiment, the intestinal polyp is an upper intestinal polyp or a colonic polyp. In another embodiment, the nucleic acid sample derived from intestinal tissue is derived from upper intestinal tissue or colonic tissue. In other embodiments, the gene expression product is DNA or mRNA. Preferably, when the gene expression product is DNA or mRNA, the gene expression profile is determined utilizing specific hybridization probes. For example, the gene expression profile may be determined utilizing oligonucleotide microarrays.

In another embodiment, the gene expression product is a polypeptide. Preferably, when the gene expression product is a polypeptide, the gene expression profile is determined utilizing antibodies.

In still another embodiment, the one or more informative genes is selected from the group consisting of apoptosis genes, cell cycle genes, tumor suppressor genes, cell adhesion genes, transcription related genes, and inflammation genes. In yet another embodiment, the one or more informative genes is selected from the group consisting of the genes in FIGS. 1A-1U.

The invention also features a method of identifying an intestinal polyp, comprising obtaining a polypeptide sample derived from intestinal tissue; and determining a gene expression profile from a gene expression product of at least one informative gene having decreased expression in an intestinal polyp relative to a control, where the gene expression product is a polypeptide. Decreased expression of the gene expression product in the sample is indicative of an intestinal polyp. In one embodiment, the intestinal polyp is an upper intestinal polyp or a colonic polyp. In another embodiment, the polypeptide sample derived from intestinal tissue is derived from upper intestinal tissue or colonic tissue. In another embodiment, the gene expression profile is determined utilizing antibodies. In yet another embodiment, the one or more informative genes is selected from the group consisting of apoptosis genes, cell cycle genes, tumor suppressor genes, cell adhesion genes, transcription related genes, and inflammation genes. In still another embodiment, the one or more informative genes is selected from the group consisting of the genes in FIGS. 1A-1U.

The invention also features a method of identifying a compound for use in modulating intestinal polyp development, comprising the steps of: a) providing a cell or cell lysate sample; b) contacting the cell or cell lysate sample with a candidate compound; and c) detecting an increase in expression of at least one informative gene having decreased expression in an intestinal polyp. A candidate compound that increases the expression of the informative gene is a compound for use in modulating intestinal polyp development. In one embodiment, the intestinal polyp is an upper intestinal poly or a colonic polyp. In another embodiment, the cell or cell lysate sample is derived from intestinal tissue. The intestinal tissue may be derived from upper intestinal tissue or colonic tissue. In another embodiment, the cell or cell lysate sample is derived from a cultured cell. In other embodiments, gene expression is determined by assessing the DNA or mRNA level of the gene. Preferably, the DNA or mRNA level is determined utilizing specific hybridization probes. For example, the DNA or mRNA level may be determined utilizing oligonucleotide microarrays.

In another embodiment, gene expression is determined by assessing the polypeptide level encoded by the informative gene. Preferably, gene expression is determined utilizing antibodies.

In another embodiment, the one or more informative genes is selected from the group consisting of apoptosis genes, cell cycle genes, tumor suppressor genes, cell adhesion genes, transcription related genes, and inflammation genes. In a preferred embodiment, the one or more informative genes is selected from the group consisting of the genes in FIGS. 1A-1U.

In addition, the invention features a method of identifying a compound for use in modulating intestinal polyp development, comprising the steps of: a) providing a cell or cell lysate sample; b) contacting the cell or cell lysate sample with a candidate compound; and c) detecting a decrease in expression of at least one informative gene having increased expression in an intestinal polyp. A candidate compound that decreases the expression of the informative gene is a compound for use in modulating intestinal polyp development. In one embodiment, the intestinal polyp is an upper intestinal poly or a colonic polyp. In another embodiment, the cell or cell lysate sample is derived from intestinal tissue. The intestinal tissue may be derived from upper intestinal tissue or colonic tissue. In another embodiment, the cell or cell lysate sample is derived from a cultured cell. In other embodiments, gene expression is determined by assessing the DNA or mRNA level of the gene. Preferably, the DNA or mRNA level is determined utilizing specific hybridization probes. For example, the DNA or mRNA level may be determined utilizing oligonucleotide microarrays.

In another embodiment, gene expression is determined by assessing the polypeptide level encoded by the informative gene. Preferably, gene expression is determined utilizing antibodies.

In another embodiment, the one or more informative genes is selected from the group consisting of apoptosis genes, cell cycle genes, tumor suppressor genes, cell adhesion genes, transcription related genes, and inflammation genes. In a preferred embodiment, the one or more informative genes is selected from the group consisting of the genes in FIGS. 1A-1U.

The invention also features a method for modulating intestinal polyp development in a subject by down-regulating in the subject at least one informative gene shown to be expressed in intestinal polyp tissue or expressed in increased levels, but not in normal intestinal tissue.

The invention also features a method for modulating intestinal polyp development in a subject by up-regulating in the subject at least one informative gene shown not to be expressed in intestinal polyp samples, or expressed in reduced levels relative to normal intestinal samples.

The invention also features an oligonucleotide microarray having immobilized thereon a plurality of oligonucleotide probes specific for one or more informative genes selected from the group consisting of the genes on FIGS. 1A-1U.

The invention also features an oligonucleotide microarray having immobilized thereon a plurality of oligonucleotide probes specific for one or more informative genes selected from the group consisting of apoptosis genes, cell cycle genes, tumor suppressor genes, cell adhesion genes, transcription related genes, and inflammation genes.

### BRIEF DESCRIPTION OF THE DRAWINGS

FIGS. 1A-1V show the results of differential expression studies described herein. FIGS. 1A-1U are portions that complete a table listing the nucleic acid molecules which are differentially expressed in intestinal polyps and normal intestinal tissue. The first column of the table shows the nucleic acid molecule name, and the second column shows the Affymetrix annotation. Columns 3-6 show expression data for the colonic polyp samples (L1-L4; gray), and Columns 7-10 show expression data for the normal colon samples (LC1-LC4). Columns 11-14 show expression data for the intestinal polyp samples (U1-U4; gray), and Columns 15-18 show expression data for the normal intestinal samples (UC1-UC4). Columns 19-20, 21-22, 23-24, and 25-26 show the average expression value and standard deviation, respectively, for the groups of colonic expression, small intestine expression, polyp expression, and normal tissue expression, respectively. Column 27 shows a short description of the function of the nucleic acid molecule shown in Column 1 (if available). FIG. 1V provides a key depicting how FIGS. 1A-1U are assembled to produce a complete table.

### DETAILED DESCRIPTION OF THE INVENTION

The present invention is directed to methods for predicting phenotypic classes of intestinal polyps, such as the presence or absence of an intestinal polyp, or the identification of compounds that modulate intestinal polyp development, based on gene expression profiles. In one aspect, the invention involves identifying an intestinal polyp by obtaining a nucleic acid or polypeptide sample derived from intestinal tissue, and determining a gene expression profile from a gene expression product of at least one informative gene having increased expression in an intestinal polyp relative to a control. Increased expression of the informative gene is indicative of the presence of an intestinal polyp in the sample. Alternatively, identification of a polyp in a sample may occur by obtaining a nucleic acid or polypeptide sample derived from intestinal tissue, and determining a gene expression profile from a gene expression product of at least one informative gene having decreased expression in an intestinal polyp relative to a control. Decreased expression of the informative gene is indicative of the presence of an intestinal polyp in the sample.

As used herein, by “intestinal polyp” is meant abnormal cell growth in the intestines. Intestinal polyps can form in the upper intestines (for example, the small intestines) or the large intestines (also known as the colon). Intestinal polyps may be benign polyps (also referred to as non-neoplastic, hyperplastic, or inflammatory polyps), which do not appear to have the potential to develop into neoplastic polyps, or they may be malignant polyps (also referred to as neoplastic polyps, adenomas, tubular adenomas, tubulovillous adenomas or villoglandular polyps). In addition, intestinal cancers can arise from previously benign polyps.

By “presence of an intestinal polyp” is meant that a sample, for example, a tissue sample contains an intestinal polyp or a cancerous intestinal polyp, or that the sample is at risk for, or has a likelihood of developing an intestinal polyp or a cancerous intestinal polyp.

By “absence of an intestinal polyp” is meant that a sample, for example, a tissue sample does not contain an intestinal polyp or a cancerous intestinal polyp, or that the sample has a decreased risk of, or has a decreased likelihood of developing an intestinal polyp or a cancerous intestinal polyp.

By “gene expression profile” is meant the level or amount of gene expression of particular genes, for example, informative genes, as assessed by methods described herein. The gene expression profile can comprise data for one or more informative genes and can be measured at a single time point or over a period of time. For example, the gene expression profile can be determined using a single informative gene, or it can be determined using two or more informative genes, three or more informative genes, five or more informative genes, ten or more informative genes, twenty-five or more informative genes, or fifty or more informative genes. A gene expression profile may include expression levels of genes that are not informative, as well as informative genes. Phenotype classification (e.g., the presence or absence of an intestinal polyp, or the identification of a compound that modulates intestinal polyp development) can be made by comparing the gene expression profile of the sample with respect to one or more informative genes with one or more gene expression profiles (e.g., in a database). Using the methods described herein, expression of numerous genes can be measured simultaneously. The assessment of numerous genes provides for a more accurate evaluation of the sample because there are more genes that can assist in classifying the sample. A gene expression profile may involve only those genes that are increased in expression in a sample, only those genes that are decreased in expression in a sample, or a combination of genes that are increased and decreased in expression in a sample.

As used herein, “informative genes,” refers to a gene or genes whose expression correlates with a particular phenotype. Expression profiles obtained for informative genes can be used to determine, for example, the presence or absence of an intestinal polyp in a sample derived from intestinal tissue, or if a candidate compound increases or decreases gene expression in a sample. Samples can be classified according to their broad expression profile, or according to the expression levels of particular informative genes. The genes that are relevant for classification are referred to herein as “informative genes.” Not all informative genes for a particular class distinction must be assessed in order to classify a sample. Similarly, the set of informative genes that characterize one phenotypic effect may or may not be the same as the set of informative genes for a different phenotypic effect. For example, a subset of the informative genes that demonstrate a high correlation with a class distinction can be used in classifying the presence of an intestinal polyp. This subset can be, for example, one or more genes, two or more genes, three or more genes, five or more genes, ten or more genes, twenty-five or more genes, or fifty or more genes. The informative genes that characterize other classification categories such as, for example, a candidate compound that modulates intestinal polyp development, can be the same or different from the informative genes that characterize the presence or absence of an intestinal polyp. Typically the accuracy of the classification increases with the number of informative genes that are assessed.

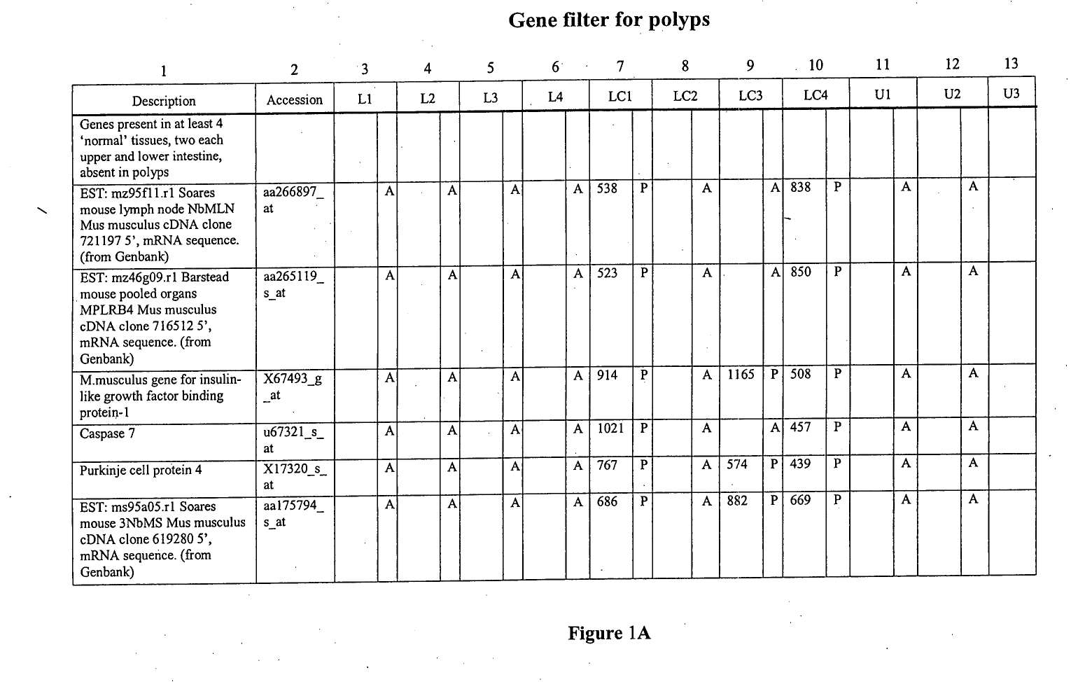

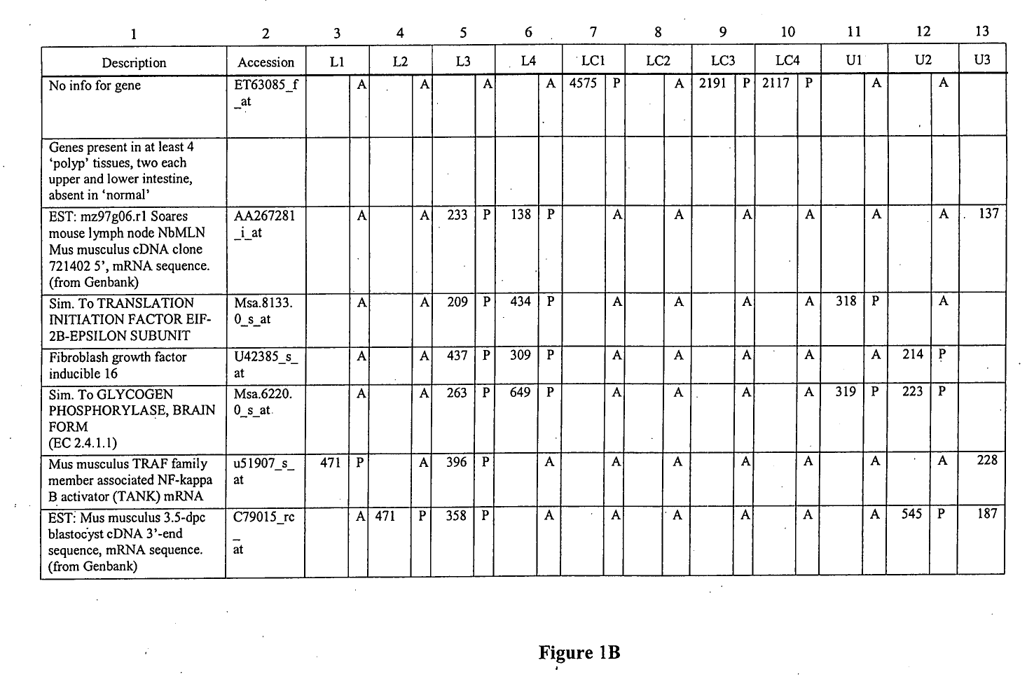

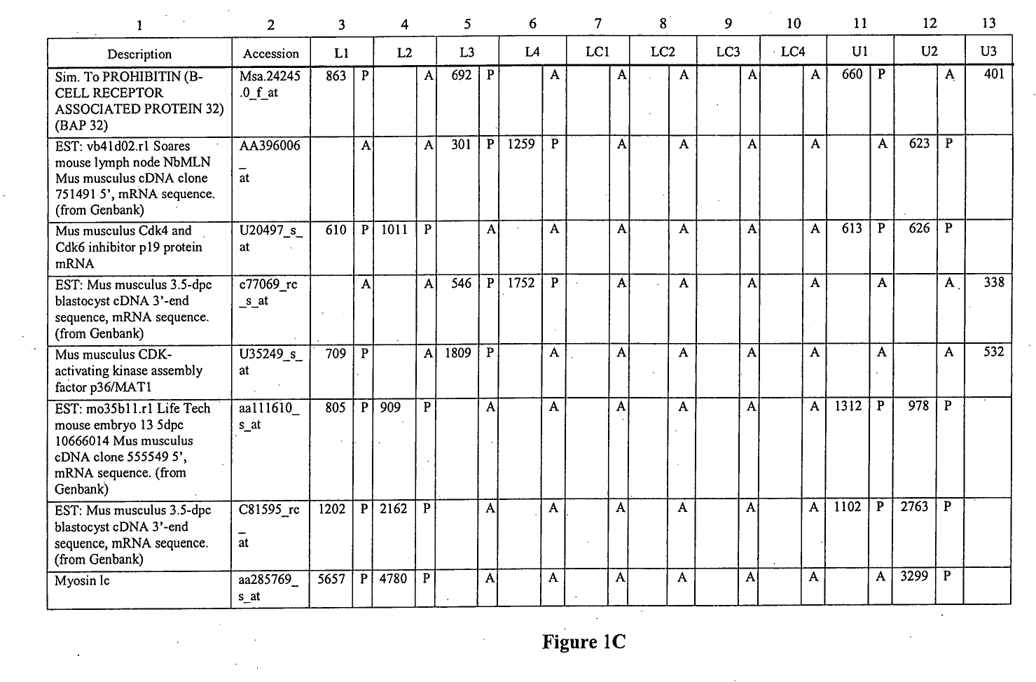

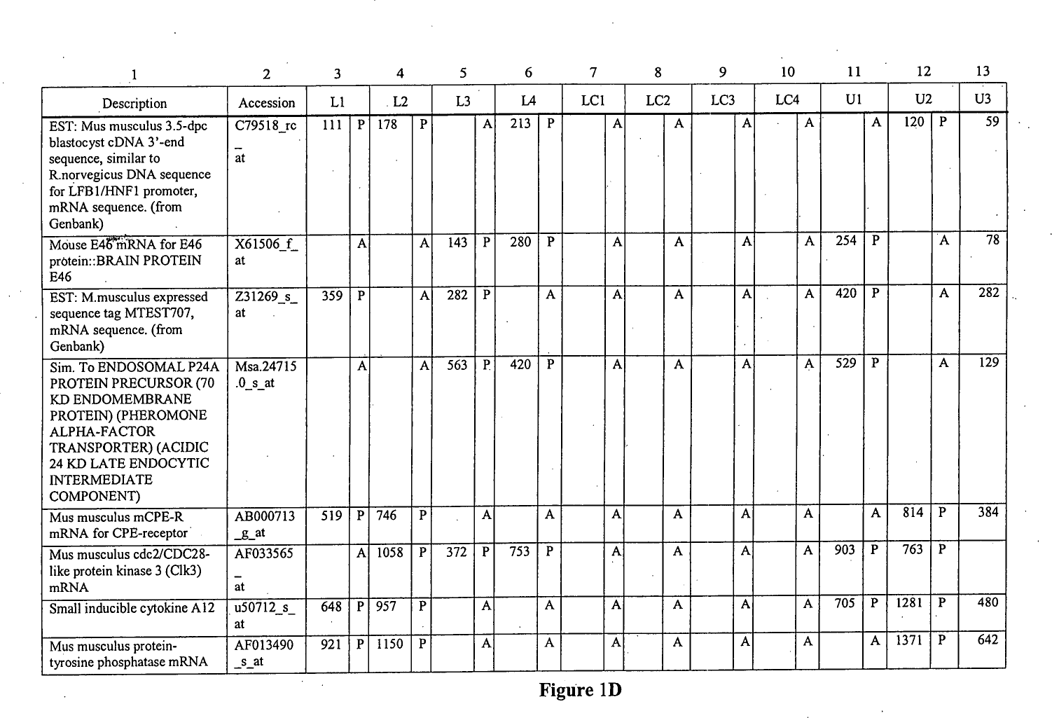

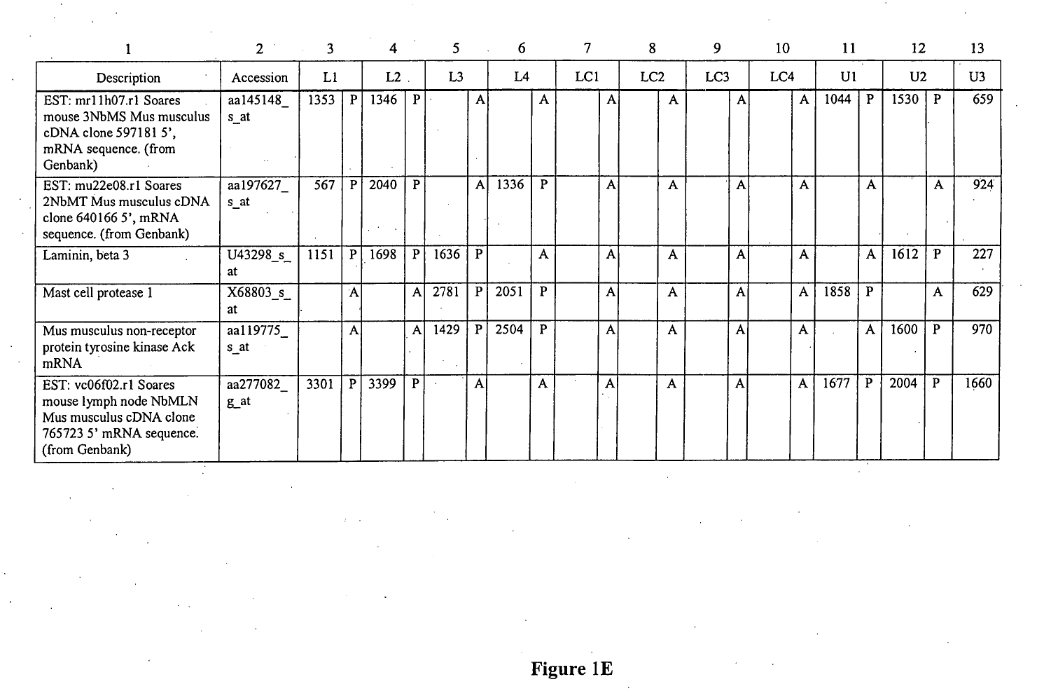

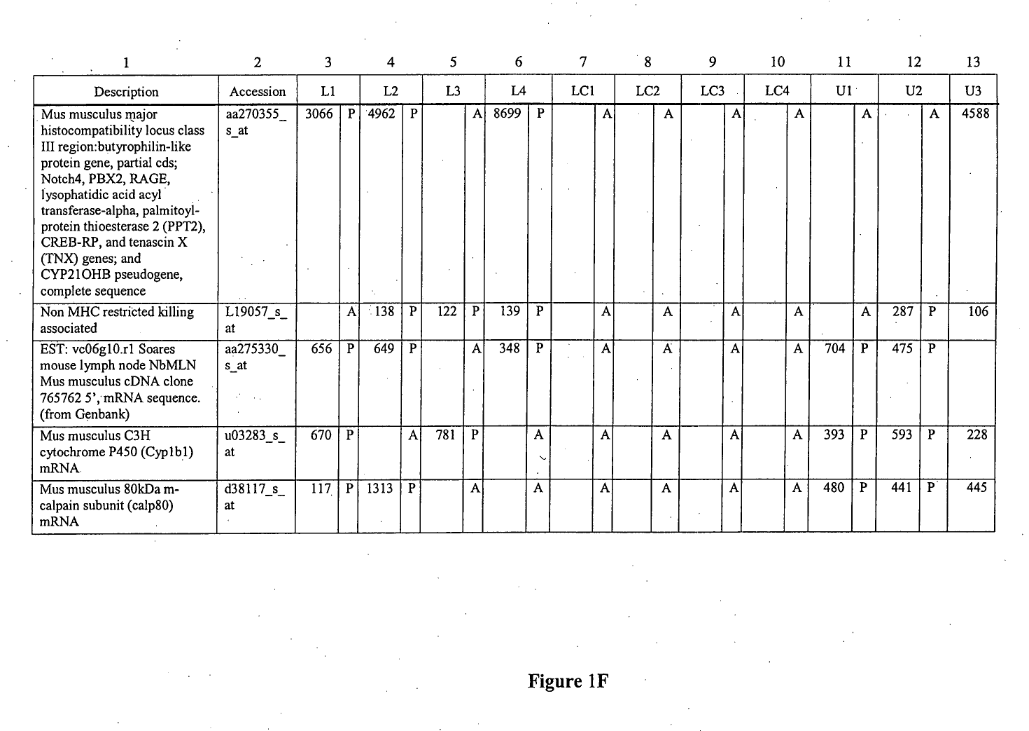

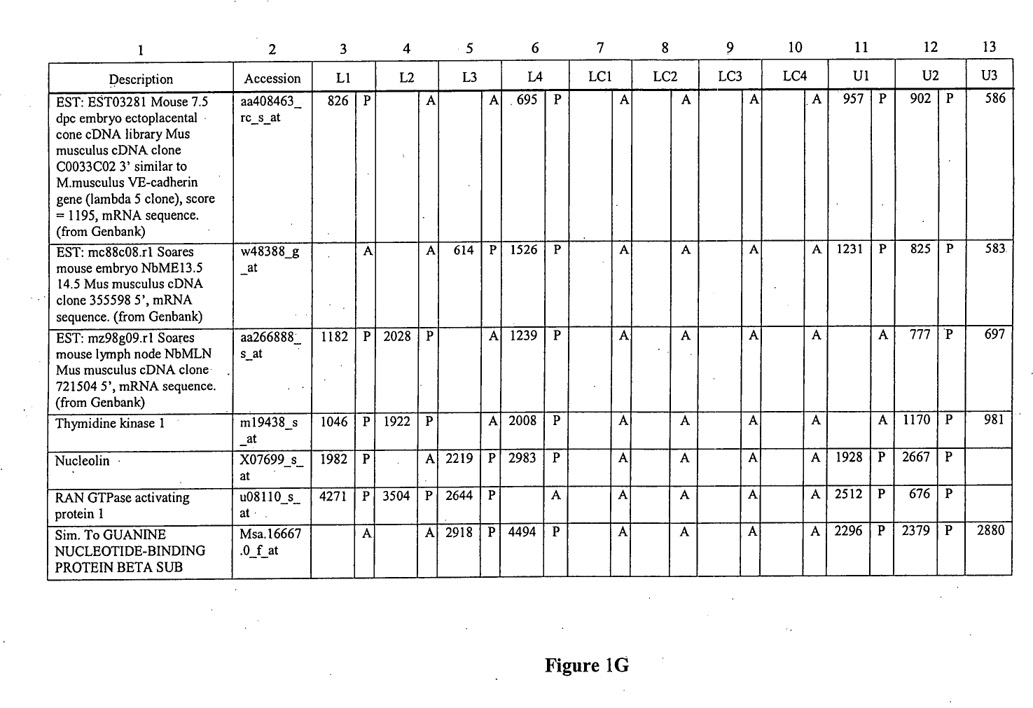

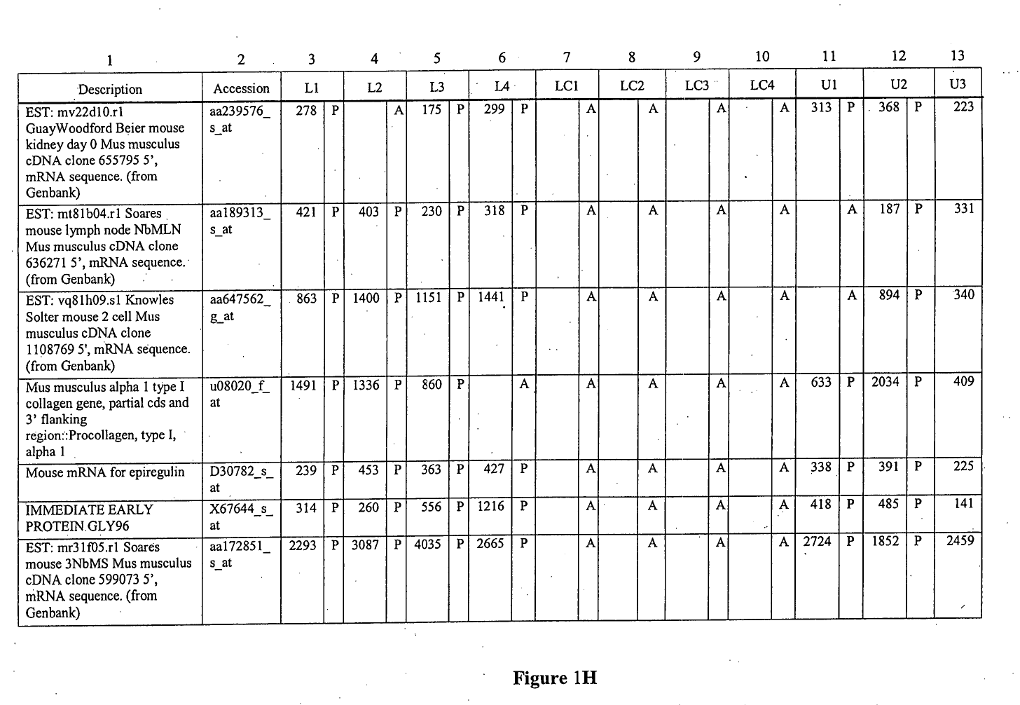

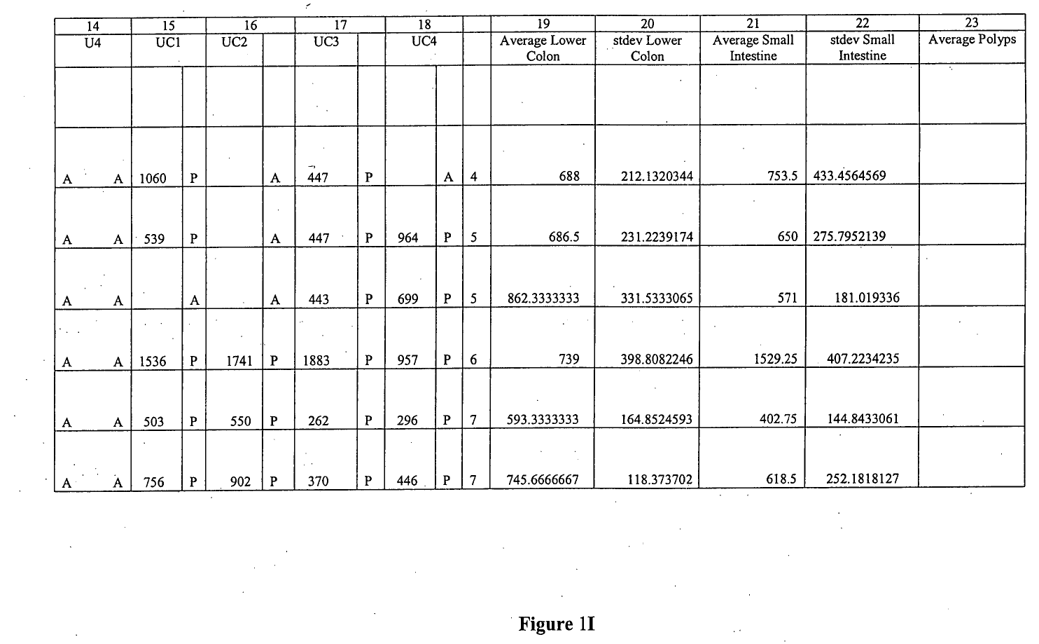

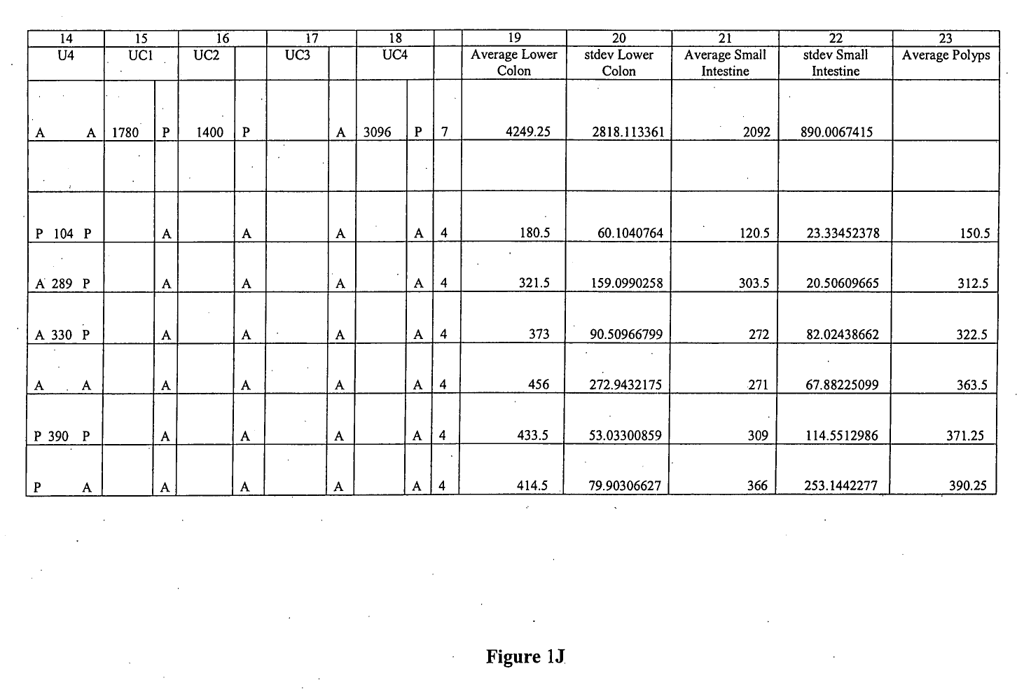

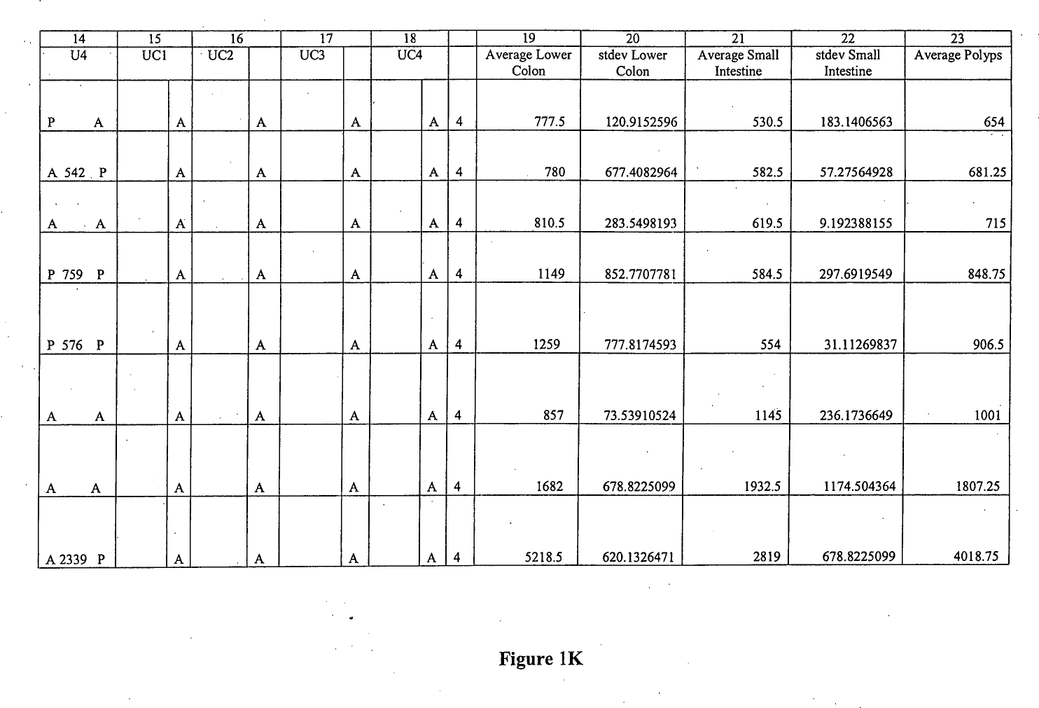

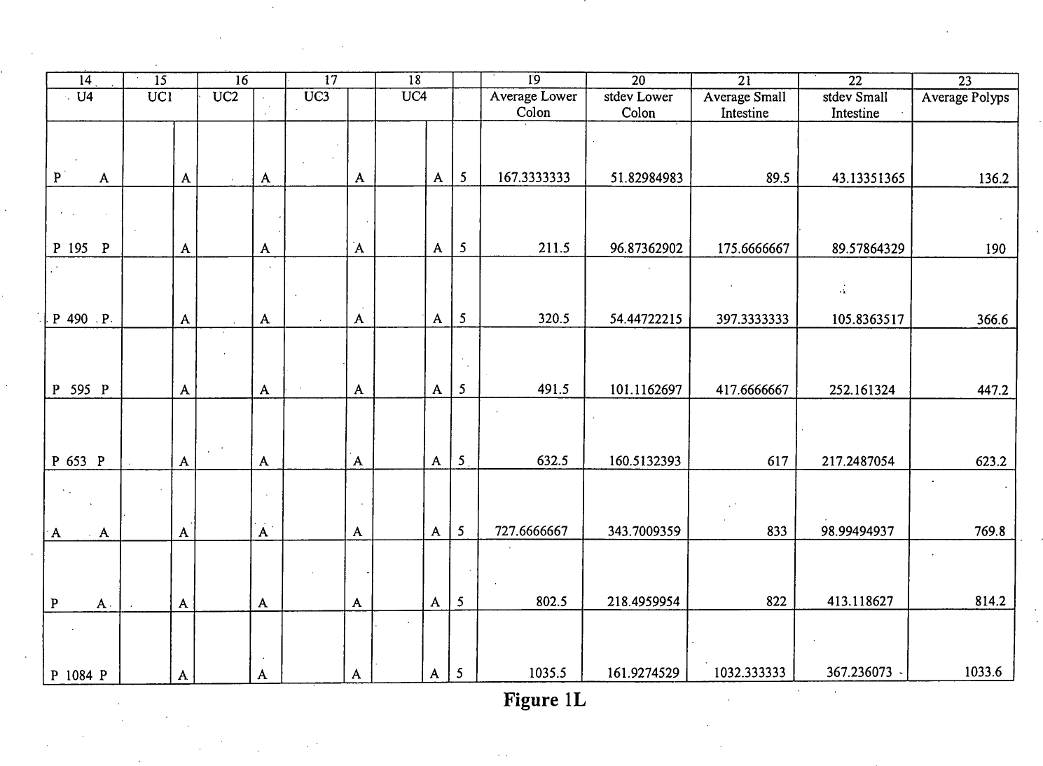

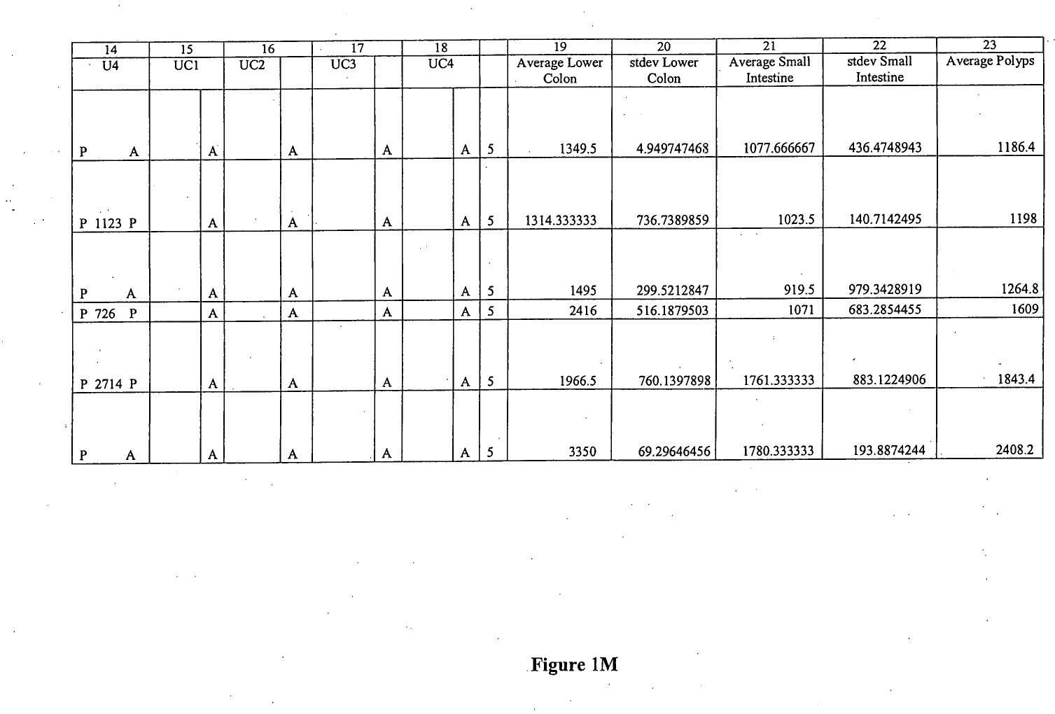

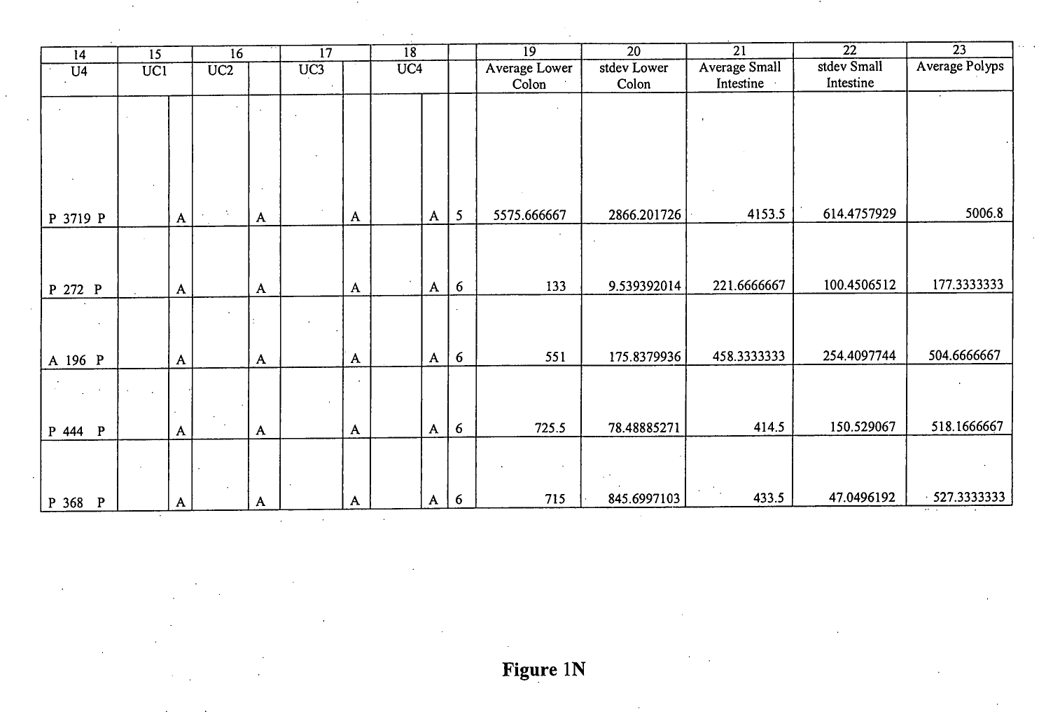

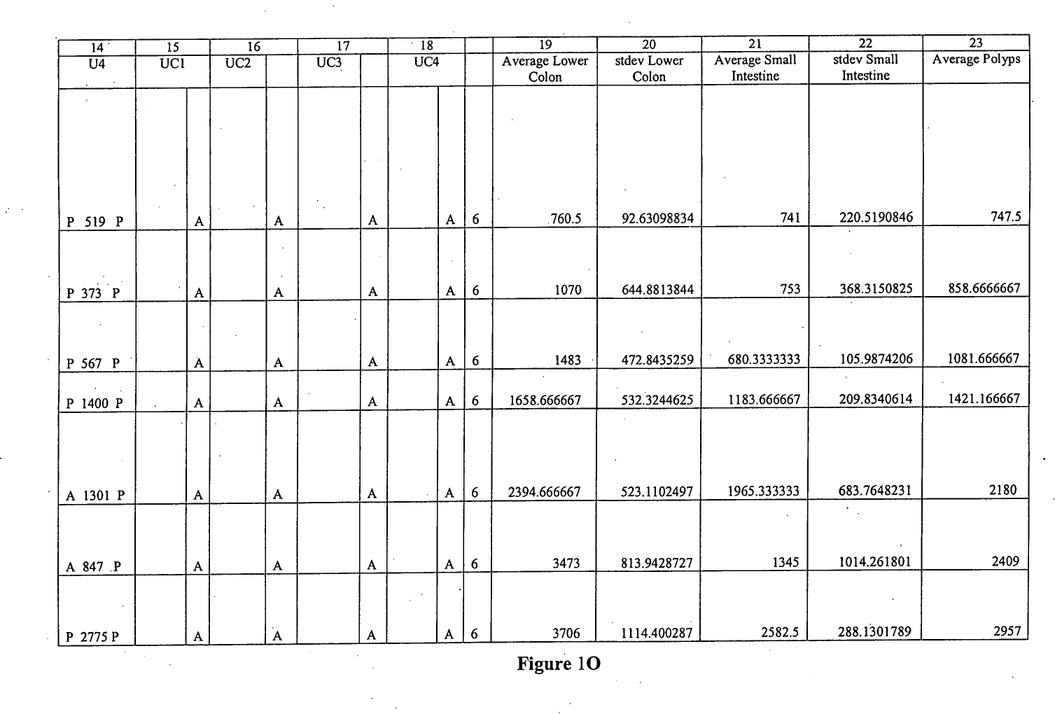

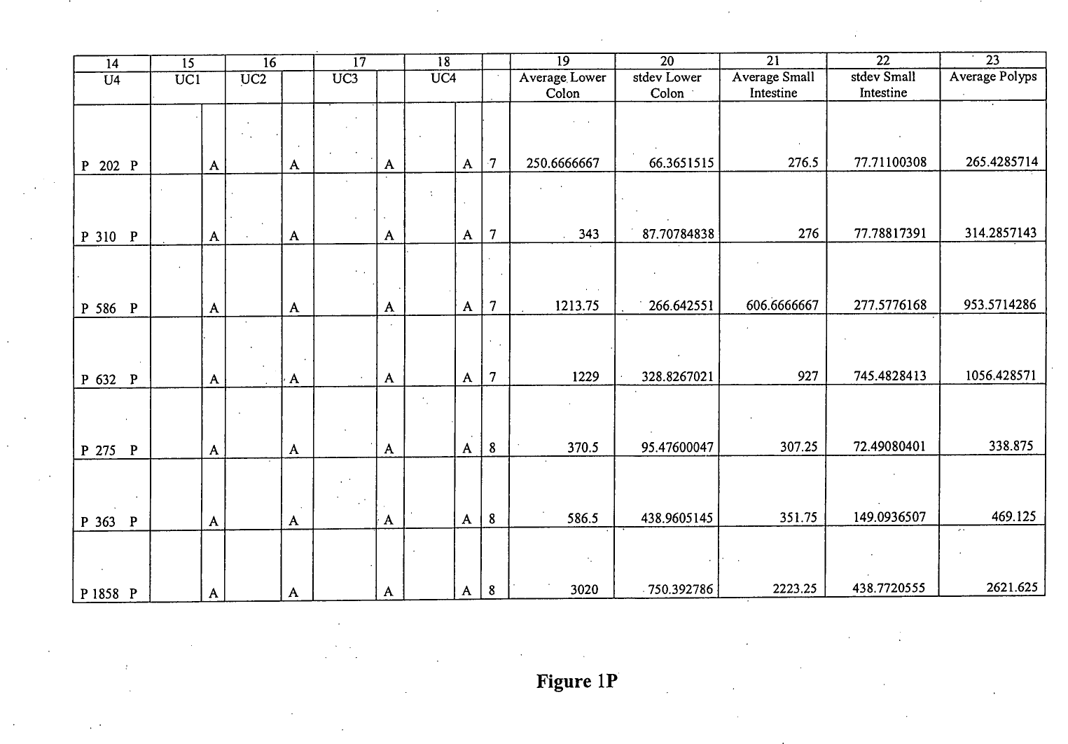

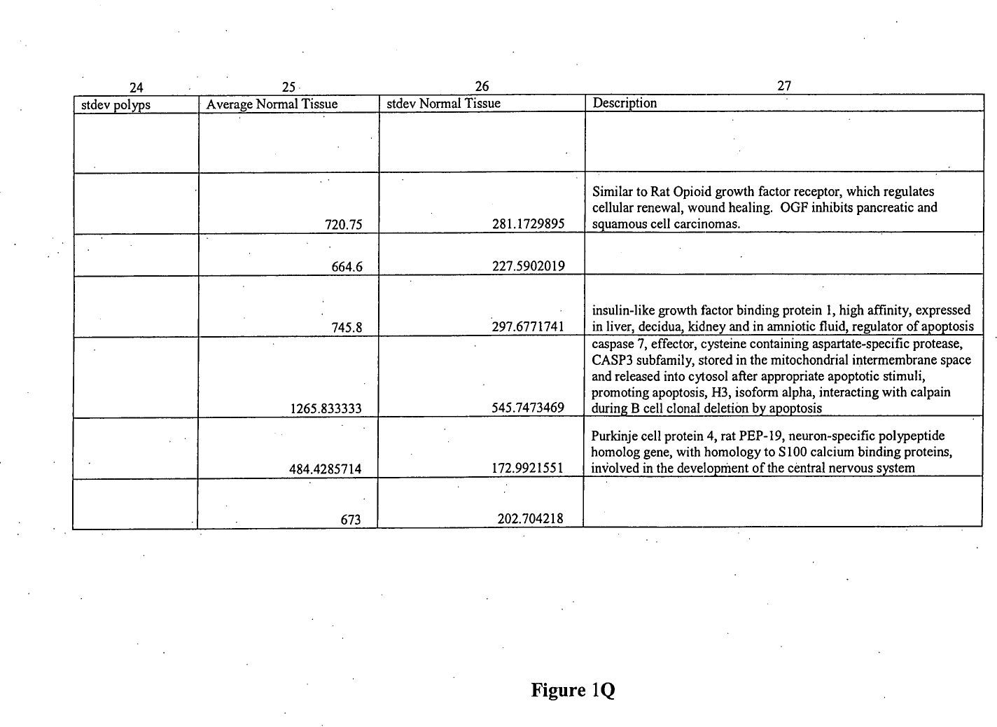

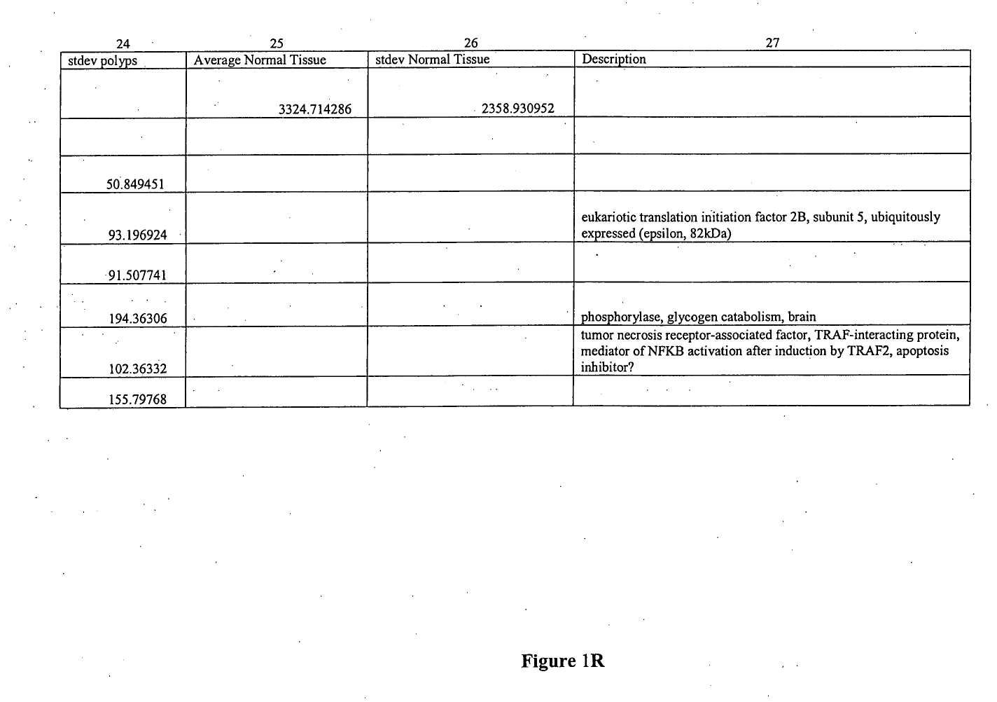

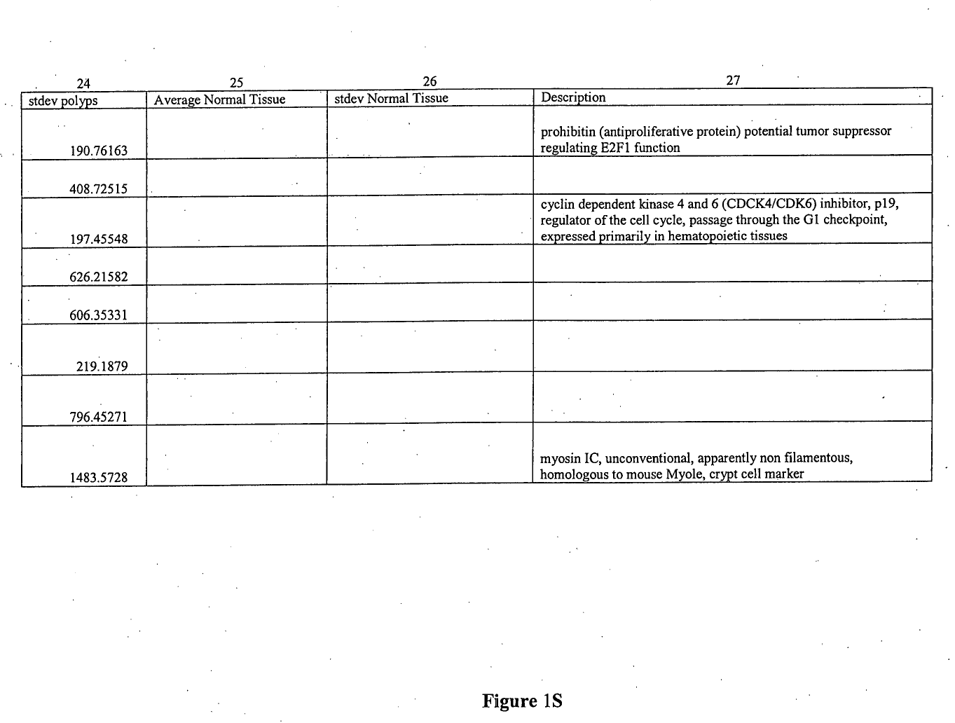

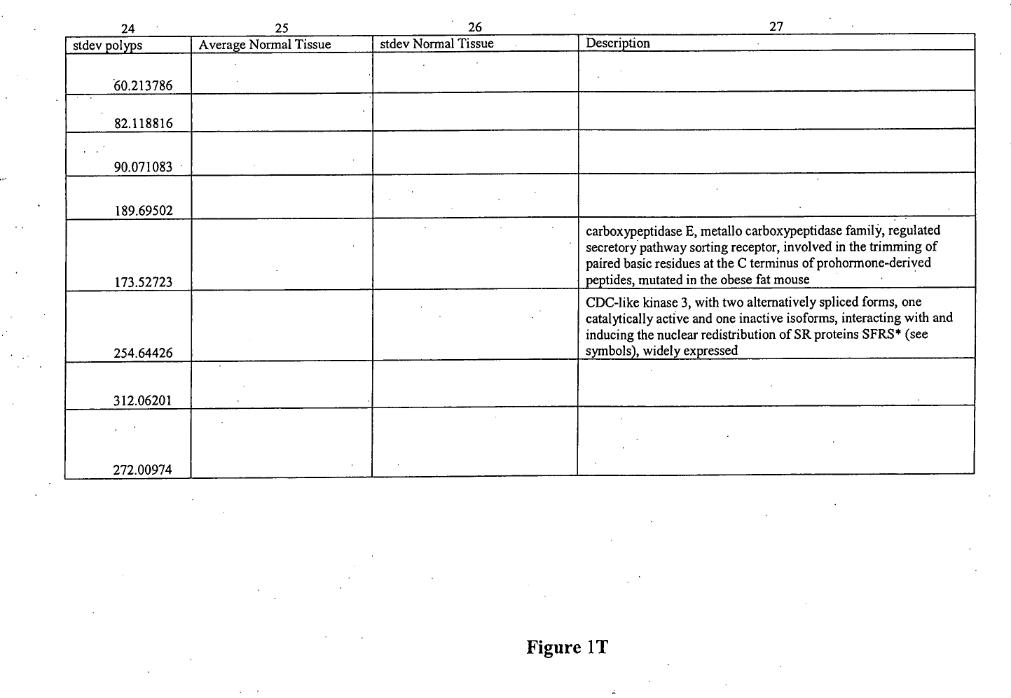

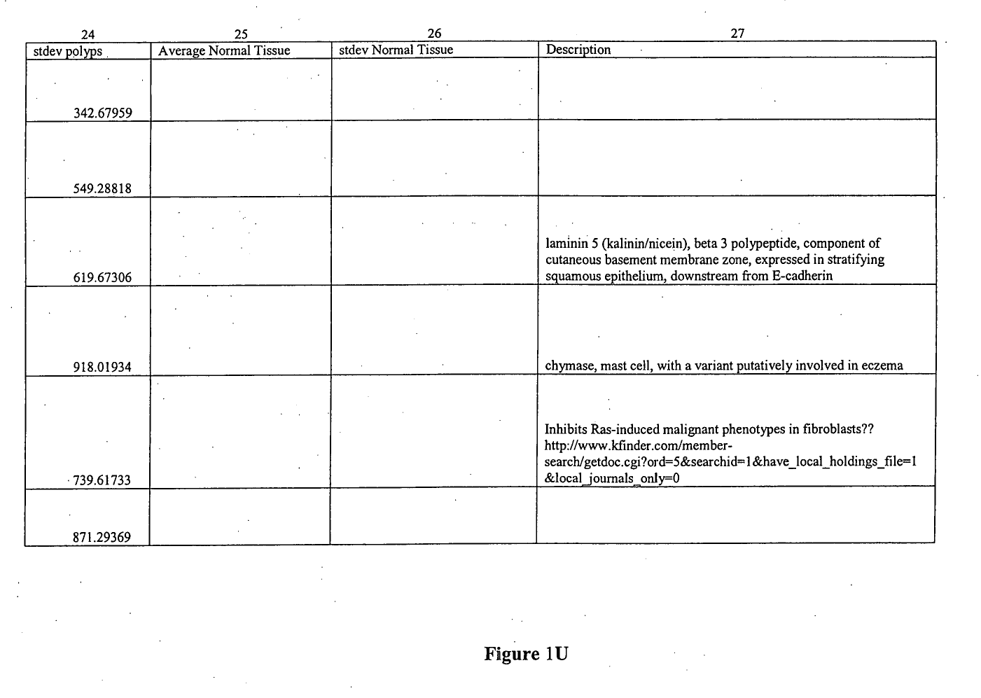

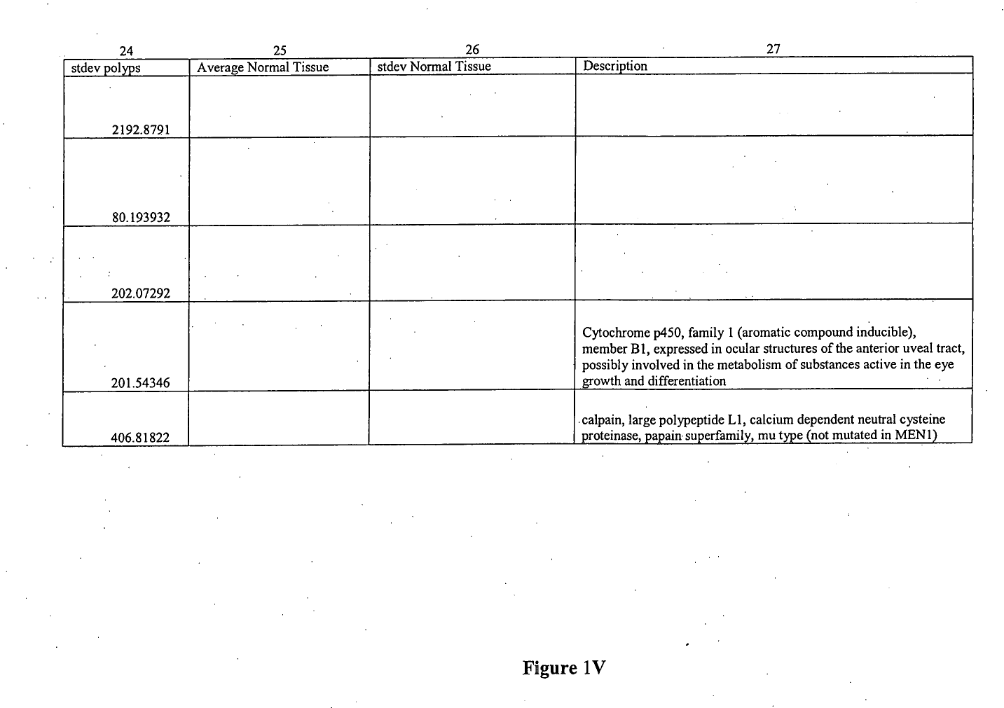

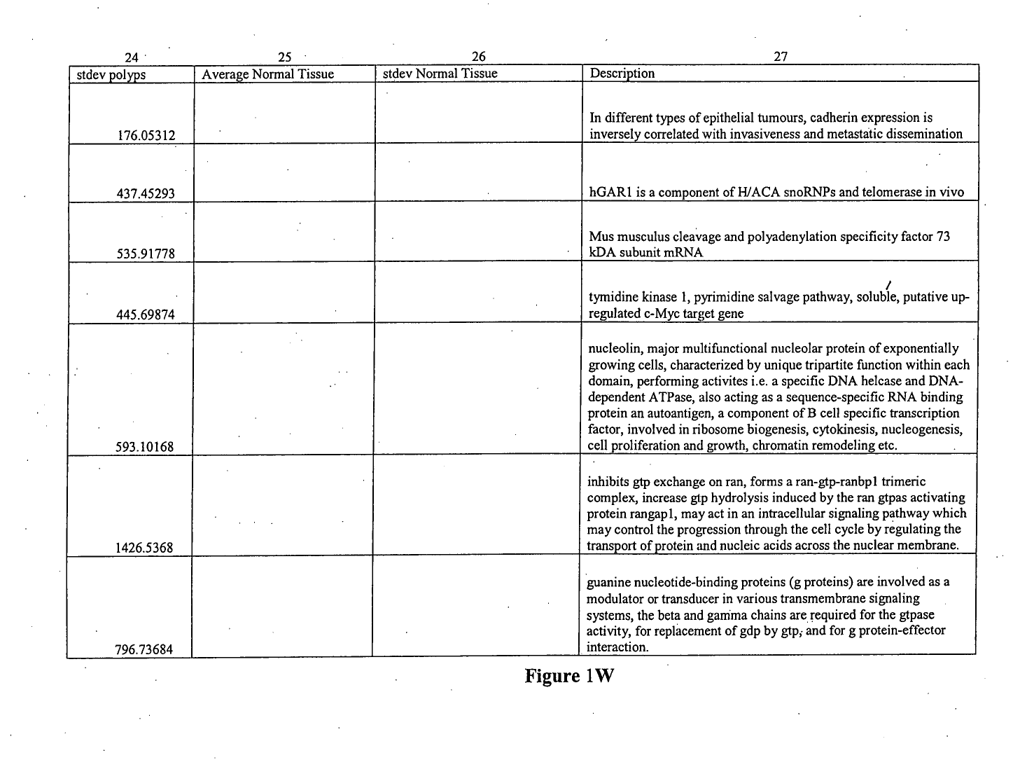

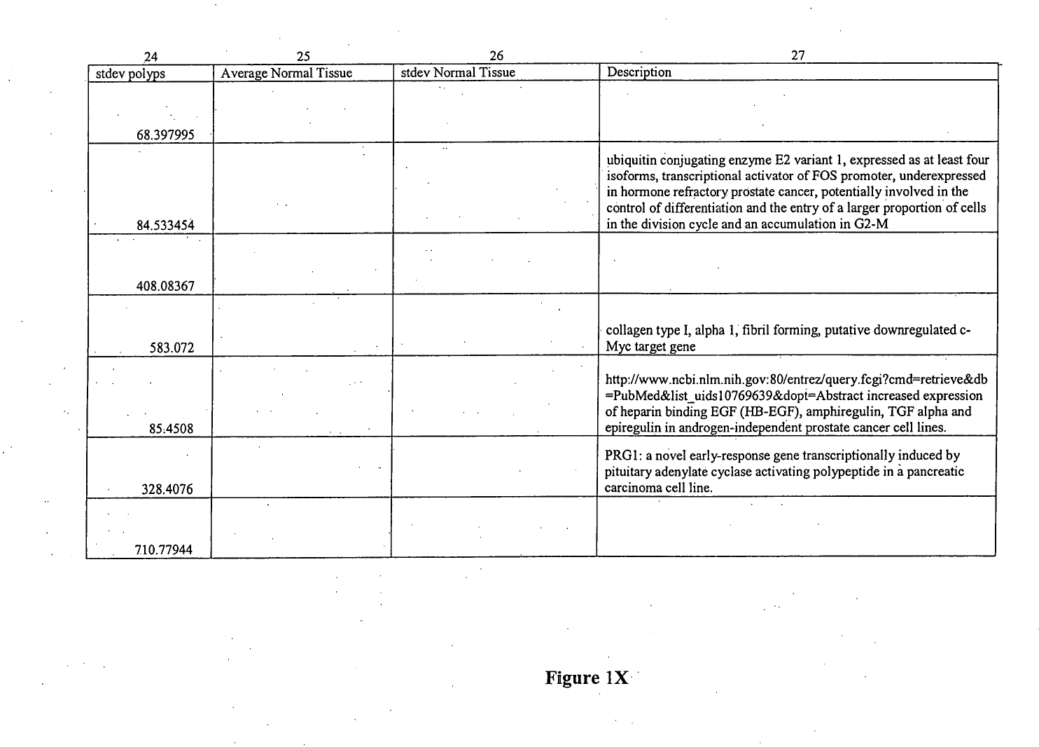

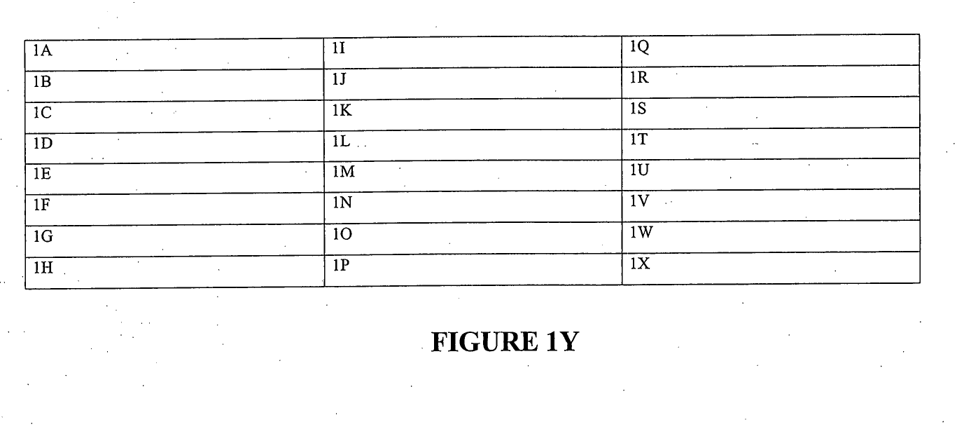

Informative genes include, but are not limited to, apoptosis genes, cell cycle genes, tumor suppressor genes, cell adhesion genes, transcription related genes, inflammation genes, as well as the particular genes shown in FIGS. 1A-1U.

By an “apoptosis gene” is meant a gene or nucleic acid that encodes a polypeptide involved in the control of apoptosis. The apoptosis gene may be a gene involved in the promotion of apoptosis, or the apoptosis gene may be a gene involved in preventing apoptosis. Examples of apoptosis genes include, but are not limited to: TANK1 and Epiregulin.

By a “cell cycle gene” is meant a gene or nucleic acid that encodes a polypeptide involved in the control of the cell cycle. The cell cycle gene may be a gene involved in speeding up, slowing down, or arresting any phase of the cell cycle. Examples of cell cycle genes include, but are not limited to: CDK4/CDK6 inhibitor, RAN GTPase activating protein 1, and ubiquitin conjugating enzyme E2 variant 1.

By a “tumor suppressor gene” is meant is meant a gene or nucleic acid that encodes a polypeptide involved in decreasing or preventing tumor formation, development, or progression. Examples of tumor suppressor genes include, but are not limited to: prohibitin, non-receptor protein tyrosine kinase Ack, and PRG1.

By a “cell adhesion gene” is meant a gene or nucleic acid that encodes a polypeptide involved in the control of cell adhesion. The cell adhesion gene may be a gene involved increasing cell adhesion properties or decreasing cell adhesion properties. In addition, the cell adhesion gene may be involved in mediating cell-cell adhesion, or cell-extracellular matrix adhesion. Examples of cell adhesion genes include, but are not limited to: collagen type 1α, E-cadherin, and Laminin β3.

By a “transcription related gene” is meant a gene or nucleic acid that encodes a polypeptide involved in the control of transcription and/or translation. The transcription related gene may be a gene involved in, for example, transcription initiation, translation initiation, ribosome biogenesis, cytokinesis, chromatin remodeling, splicing, pre-rRNA processing, or telomerase formation. Examples of transcription related genes include, but are not limited to: Translation Initiation Factor EIF-2B-ε subunit, cleavage and polyadenylation specificity factor 73 kDa subunit, Nucleolin, cdc2/CDC28-like protein kinase 3 (Clk3), and hGAR1.

By an “inflammation gene” is meant a gene or nucleic acid that encodes a polypeptide involved in the control of a localized protective response elicited by injury or destruction of tissues, which serves to destroy, dilute or sequester both the injurious agent and the injured tissue. Inflammation is characterized in the acute form by the classical signs of pain, heat, redness, swelling, and loss of function. Histologically, inflammation involves a complex series of events, including dilatation of arterioles, capillaries, and venules, with increased permeability and blood flow; exudation of fluids, including plasma proteins; and leukocytic migration into the inflammatory focus. Inflammation genes may be found in a number of different cells, including cells of the immune system, for example, mast cells. Examples of inflammation genes include, but are not limited to chymase.

As used herein, “gene expression products” are proteins, polypeptides, or nucleic acid molecules (e.g., mRNA, tRNA, rRNA, cDNA, or cRNA) that result from transcription or translation of genes. The present invention can be used effectively to analyze proteins, polypeptides, or nucleic acid molecules that are the result of transcription or translation, particularly of informative genes identified herein. The nucleic acid molecule levels measured can be derived directly from the gene or, alternatively, from a corresponding regulatory gene or regulatory sequence element. All forms of gene expression products can be measured. For example, the nucleic acid molecule can be transcribed to obtain an RNA gene expression product. If desired, the transcript can be translated using, for example, standard in vitro translation methods to obtain a polypeptide gene expression product. Polypeptide gene expression products can be used in protein binding assays, for example, antibody assays, or in nucleic acid binding assays, standardly known in the art, in order to identify intestinal polyps or compounds involved in polyp development. Additionally, variants of genes and gene expression products including, for example, spliced variants and polymorphic alleles, can be measured. Similarly, gene expression can be measured by assessing the level of a polypeptide or protein or derivative thereof translated from mRNA. The sample to be assessed can be any sample that contains a gene expression product. Suitable sources of gene expression products, e.g., samples, can include intact cells, lysed cells, cellular material for determining gene expression, or material containing gene expression products. Examples of such samples are intestinal tissue, cells derived from intestinal tissue, nucleic acids or polypeptides derived from intestinal tissue, blood, plasma, lymph, urine, tissue, mucus, sputum, saliva, or other cell samples. Methods of obtaining such samples are known in the art.

By “increased expression” is meant the level of a gene expression product is made higher and/or the activity of the gene expression product is enhanced. Preferably, the increase is by at least 1.5-fold, more preferably the increase is at least 2-fold, 5-fold, or 10-fold, and most preferably, the increase is at least 20-fold, relative to a control. In the work described herein, a gene was considered to have increased expression in an intestinal polyp if it was expressed in at least 4 out of 8 polyp tissue samples (at least 2 of which were colonic and 2 of which were intestinal) and absent in all normal tissues.

By “decreased expression” is meant the level of a gene expression product is made lower and/or the activity of the gene expression product is lowered. Preferably, the decrease is at least 25%, more preferably, the decrease is at least 50%, 60%, 70%, 80%, or 90% and most preferably, the decrease is at least one-fold, relative to a control sample. In the work described herein, a gene was considered to have decreased expression if it was expressed in at least 4 out of 8 normal tissue samples (at least 2 of which were colonic and 2 of which were intestinal) and absent in all polyps.

Genes that are particularly relevant for classification, i.e., demonstrate a different expression profile in different classification categories, have been identified as a result of work described herein and are shown in FIGS. 1A-1U.

In one embodiment, the gene expression product is a protein or polypeptide. As used herein, by “polypeptide” is meant any chain of more than two amino acids, regardless of post-translational modification such as glycosylation or phosphorylation. Examples of polypeptides include, but are not limited to, proteins. In this embodiment the determination of the gene expression profile is made using techniques for protein detection and quantitation known in the art. For example, antibodies that specifically interact with the protein or polypeptide expression product of one or more informative genes can be obtained using methods that are routine in the art. The specific binding of such antibodies to protein or polypeptide gene expression products can be detected and measured by methods known in the art, for example, Western blot analysis or ELISA techniques.

In a preferred embodiment, the gene expression product is a nucleic acid, for example, DNA or mRNA, and the gene expression levels are obtained by contacting the sample with a suitable microarray on which probes specific for all or a subset of the informative genes have been immobilized, and determining the extent of hybridization of the nucleic acid in the sample to the probes on the microarray. Such microarrays are also within the scope of the invention. Examples of methods of making oligonucleotide microarrays are described, for example, in WO 95/11995. Other methods are readily known to the skilled artisan.

The gene expression value measured or assessed is the numeric value obtained from an apparatus that can measure gene expression levels. Gene expression levels refer to the amount of expression of the gene expression product, as described herein. The values are raw values from the apparatus, or values that are optionally re-scaled, filtered and/or normalized. Such data is obtained, for example, from a GeneChip® probe array or Microarray (Affymetrix, Inc.; U.S. Pat. Nos. 5,631,734, 5,874,219, 5,861,242, 5,858,659, 5,856,174, 5,843,655, 5,837,832, 5,834,758, 5,770,722, 5,770,456, 5,733,729, 5,556,752, all of which are incorporated herein by reference in their entirety), and the expression levels are calculated with software (e.g., Affymetrix GENECHIP software). For example, nucleic acids (e.g., mRNA or DNA) from a sample that has been subjected to particular stringency conditions hybridize to the probes on the chip. The nucleic acid to be analyzed (e.g., the target) is isolated, amplified and labeled with a detectable label, (e.g., ³²P or fluorescent label) prior to hybridization to the arrays. After hybridization, the arrays are inserted into a scanner that can detect patterns of hybridization. These patterns are detected by detecting the labeled target now attached to the microarray, e.g., if the target is fluorescently labeled, the hybridization data are collected as light emitted from the labeled groups. Since labeled targets hybridize, under appropriate stringency conditions known to one of skill in the art, specifically to complementary oligonucleotides contained in the microarray, and since the sequence and position of each oligonucleotide in the array are known, the identity of the target nucleic acid applied to the probe is determined.

Quantitation of gene profiles from the hybridization of a labeled nucleic acid microarray can be performed by scanning the microarray to measure the amount of hybridization at each position on the microarray with an Affymetrix scanner (Affymetrix, Santa Clara, Calif.). For each stimulus a time series of nucleic acid levels (C={C1,C2,C3, . . . Cn}) and a corresponding time series of nucleic acid levels (M={M1,M2,M3, . . . Mn}) in control medium in the same experiment as the stimulus is obtained. Quantitative data is then analyzed. Hybridization analysis using microarray is only one method for obtaining gene expression values. Other methods for obtaining gene expression values known in the art or developed in the future can be used with the present invention. Once the gene expression values are determined, the sample can be classified.

Once the gene expression levels of the sample are obtained, the levels are compared or evaluated against a model or control sample(s), and then the sample is classified, for example, based one whether a particular informative gene in the sample exhibits increased or decreased expression. The evaluation of the sample determines whether or not the sample is assigned to a particular phenotypic class, for example, whether or not the sample contains a polyp or whether or not a candidate compounds modulates intestinal polyp development.

The correlation between gene expression and class distinction can be determined using a variety of methods. Methods for defining classes and classifying samples are described, for example, in U.S. patent application Ser. No. 09/544,627, filed Apr. 6, 2000 by Golub et al., the teachings of which are incorporated herein by reference in their entirety. The information provided by the present invention, alone or in conjunction with other test results, aids in sample classification.

In a preferred correlation method, the nucleic acid molecules were considered to be expressed in normal tissue and not in polyp tissue if the nucleic acid molecule was expressed in at least 4 out of 8 normal tissue samples (at least 2 of which were colonic and 2 of which were intestinal) and absent in all polyps. Nucleic acid molecules were considered to be expressed in polyp tissue and not in normal tissue if the nucleic acid molecule was expressed in at least 4 out of 8 polyp tissue samples (at least 2 of which were colonic and 2 of which were intestinal) and absent in all normal tissues. Accordingly, a gene can be considered to have increased expression in an intestinal polyp if it is expressed in at least 4 out of 8 polyp tissue samples (at least 2 of which were colonic and 2 of which were intestinal) and absent in all normal tissues. Conversely, a gene can be considered to have decreased expression in an intestinal polyp if it is expressed in at least 4 out of 8 normal tissue samples (at least 2 of which were colonic and 2 of which were intestinal) and absent in all polyps. It should be recognized that one of skill in the art can apply more stringent or less stringent criteria in determining whether a gene expression product is increased or decreased.

The present invention also features methods for identifying compounds that modulate intestinal polyp development. Novel compounds identified as described herein are also the subject of the invention. Such methods involve contacting a sample, for example a cell, cell lysate, tissue, or tissue lysate, with a candidate compound, and detecting an increase in expression of at least one informative gene having decreased expression in an intestinal polyp. A candidate compound that increases expression of such an informative gene is a compound for use in modulating intestinal polyp development. Alternatively, a compound that modulates intestinal polyp development can be identified by contacting a sample, for example, a cell, cell lysate, tissue, or tissue lysate with a candidate compound, and detecting a decrease in expression of at least one informative gene having increased expression in an intestinal polyp. A candidate compound that decreases expression of such an informative gene is a compound for use in modulating intestinal polyp development. An increase or decrease in an informative gene may be identified using any of the methods described herein (or any analogous method known in the art). For example, oligonucleotide array systems described herein may be used to determine whether the addition of a test compound to a sample increases or decreases expression of an informative gene in that sample.

By “modulating intestinal polyp development” is meant increasing or decreasing the likelihood that an intestinal polyp will form or develop in a subject. The modulation in intestinal polyp formation may be the result of contacting a sample (for example, a cell, tissue, cell or tissue lysate, nucleic acid, or polypeptide) with a candidate compound. Preferably, the sample is derived from intestinal tissue. It will be appreciated that the degree of modulation provided by a candidate compound in a given assay will vary, but that one skilled in the art can determine the statistically significant change or a therapeutically effective change in the degree or rate of polyp development.

By “intestinal polyp development” is meant the formation or progression of an intestinal polyp. Methods for monitoring intestinal polyp development are described herein.

By a “candidate compound” is meant a molecule, be it naturally-occurring or artificially derived, that is surveyed for its effects on the gene expression profile of an informative gene, employing methods described herein. Examples of candidate compounds include, but are not limited to peptides, polypeptides, synthetic organic molecules, naturally occurring organic molecules, nucleic acid molecules, and combinations thereof.

By “increasing gene expression” is meant raising the level of expression, and/or the activity, of one or more informative genes in a cell, tissue, cell lysate, or tissue lysate sample relative to a control sample. An increase in gene expression may occur, for example, when the sample is contacted with a candidate compound for use in modulating intestinal polyp development. The control sample may be a cell, tissue, cell lysate, or tissue lysate that was not contacted with the candidate compound or that was contacted with candidate compound vehicle only. Preferably, the increase is at least 1.5-fold, more preferably the increase is at least 2-fold, 5-fold, or 10-fold, and most preferably, the increase is at least 20-fold, relative to a control sample.

By “decreasing gene expression” is meant lowering the level or expression of, and/or the activity of, one or more informative genes in a cell, tissue, cell lysate, or tissue lysate sample relative to a control sample. A decrease in gene expression may occur, for example, when the sample is contacted with a candidate compound for use in modulating intestinal polyp development. The control sample may be a cell, tissue, cell lysate, or tissue lysate that was not contacted with the candidate compound or that was contacted with candidate compound vehicle only. Preferably, the decrease in gene expression of an informative gene is at least 25%, more preferably, the decrease is at least 50%, 60%, 70%, 80%, or 90% and most preferably, the decrease is at least one-fold, relative to a control sample.

The expression level of an informative gene may be modulated by modulating transcription, translation, or mRNA or protein turnover, or the activity of the gene expression product, and such modulation may be detected using known methods for measuring mRNA and protein levels and activities, e.g., oligonucleotide microarray hybridization, RT-PCR, and ELISA and nucleic acid and protein binding assays.

A compound that increases the expression level of a gene that is decreased in an intestinal polyp can be useful for treating intestinal polyps or intestinal cancer. In addition, a compound that decreases the expression level of a gene that is increased in an intestinal polyp can also be useful for treating intestinal polyps or intestinal cancer.

While the above described candidate compound screening methods are designed primarily to identify candidate compounds that may be used to decrease intestinal polyp development, identification of candidate compounds that increase intestinal polyp development is also a feature of the present invention. Such candidate compound identification methods involve contacting a sample, for example, a cell, cell lysate, tissue, or tissue lysate with a candidate compound, and detecting an increase in expression of at least one informative gene having increased expression in an intestinal polyp. A candidate compound that increases expression of such an informative gene is a compound for use in modulating intestinal polyp development.

Alternatively, a compound that modulates intestinal polyp development can be identified by contacting a sample, for example, a cell, cell lysate, tissue, or tissue lysate with a candidate compound, and detecting a decrease in expression of at least one informative gene having decreased expression in an intestinal polyp. A candidate compound that decreases expression of such an informative gene is a compound for use in modulating intestinal polyp development. These candidate compound identification methods may be used for identifying compounds that increase intestinal polyp development or intestinal cancer, or the risk of intestinal polyp development or intestinal cancer. Such compounds may be identified as compounds to which exposure should be minimized in order to decreased one's likelihood of developing intestinal polyps or intestinal cancer.

In general, novel drugs for modulation of polyp development can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., Chembridge (San Diego, Calif.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their polyp development-modulatory activities should be employed whenever possible.

When a crude extract is found to modulate (i.e., stimulate (increase) or inhibit (decrease)) intestinal polyp development, further fractionation of the positive lead extract is desirable to isolate chemical constituents responsible for the observed effect. Thus, the goal of the extraction, fractionation, and purification process is the careful characterization and identification of a chemical entity within the crude extract having an activity that increases or deceases. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases, for example, the Min mouse model described herein, in which it is desirable to increase or decrease intestinal polyp development.

Informative genes identified as described herein can also be targeted in methods of modulating intestinal polyp formation or development. For example, expression of at least one informative gene shown to be expressed in intestinal polyp tissue or expressed in increased levels, but not in normal intestinal tissue can be down-regulated in a method of inhibiting polyp formation or development. Alternatively, expression of at least one informative gene shown not to be expressed in intestinal polyp samples, or which are expressed in reduced levels relative to normal intestinal samples can be upregulated in a method of inhibiting intestinal polyp formation or development. Compounds identified by methods described herein, for example, can be utilized in methods of treatment of intestinal polyps.

The present invention also features arrays, for example, microarrays that have a plurality of oligonucleotide probes involved in intestinal polyp development immobilized thereon. The oligonucleotide probe may be specific for one or more informative genes, selected from apoptosis genes, cell cycle genes, tumor suppressor genes, cell adhesion genes, transcription related genes, and inflammation genes and/or from those in FIGS. 1A-1U. Methods for making oligonucleotide microarrays are well known in the art, and are described, for example, in WO 95/11995, the entire teachings of which are hereby incorporated by reference.

The present invention also provides information regarding the genes that are important in intestinal polyp development, thereby providing additional targets for diagnosis and therapy. It is clear that the present invention can be used to generate databases comprising informative genes that will have many applications in medicine, research and industry; such databases are also within the scope of the invention.

The invention will be further described with reference to the following non-limiting examples. The teachings of all the patents, patent applications and all other publications and websites cited herein are incorporated by reference in their entirety.

Exemplification

Methods

A Min (Many intestinal neoplasias) mouse, which contains a mutant adenomatous polyposis coli (APC) gene and is a model for familial adenomous polyposis, was used in the procedures described herein to identify genes involved in intestinal polyp development. One Min mouse was sacrificed, and 4 polyps from the upper intestine (upper intestinal polyps) and 4 polyps from the colon (colonic polyps) were isolated. For each upper intestinal polyp, a similarly sized piece of tissue determined to be normal by microscopic evaluation was isolated from upper intestinal tissue. For each colonic polyp, a similarly sized piece of tissue determined to be normal by microscopic evaluation was isolated from colon tissue.

DNA was amplified from each of the samples utilizing an amplification procedure as described in U.S. Provisional patent application Ser. No. 60/193,708 by de Graaf et al., filed Mar. 31, 2000 and U.S. Ser. No. 09/822,789, by de Graaf et al., filed Mar. 30, 2001, the entire teachings of which are incorporated by reference. Amplified DNA was then subjected to hybridization to nucleic acid arrays obtained from Affymetrix, Inc., which contained probes for approximately 13,000 mouse genes and ESTs (GeneChip® MU11K Set, Affymetrix, Inc., Santa Clara, Calif.). Results obtained from these arrays provide a quantitative readout for expression of nucleic acid molecules within the hybridized sample.

The gene expression data obtained as described above was assessed to identify specific nucleic acid molecules (e.g., ESTs, genes) whose expression differed between polyp samples and normal tissue. Nucleic acid molecules were considered to be expressed in normal tissue and not in polyp tissue if the nucleic acid molecule was expressed in at least 4 out of 8 normal tissue samples (at least 2 of which were colonic and 2 of which were upper intestinal) and was absent in all polyps. Nucleic acid molecules were considered to be expressed in polyp tissue and not in normal tissue if the nucleic acid molecule was expressed in at least 4 out of 8 polyp tissue samples (at least 2 of which were colonic and 2 of which were upper intestinal) and was absent in all normal tissues.

Results

A listing of the nucleic acid molecules which were differentially expressed in intestinal polyp and normal intestinal tissue is shown in the table shown in FIGS. 1A-1U. FIG. 1V provides a key depicting how FIGS. 1A-1U are assembled to produce a complete table. The first column of the table shows the nucleic acid molecule name, and the second column shows the Affymetrix annotation. Columns 3-6 show expression data for the colonic polyp samples (L1-L4), and Columns 7-10 show expression data for the normal colon samples (LC1-LC4). Columns 11-14 show expression data for the intestinal polyp samples (U1-U4), and Columns 15-18 show expression data for the normal intestinal samples (UC1-UC4). Columns 19-20, 21-22, 23-24, and 25-26 show the average expression value and standard deviation, respectively, for the groups of colonic expression, small intestine expression, polyp expression, and normal tissue expression, respectively. Column 27 shows a short description of the function of the nucleic acid molecule shown in Column 1 (if available).

Genes with Decreased Expression in Intestinal Polyps

The screen identified 7 genes that were determined to be “censored” by giving exhibiting decreased expression in polyp samples. In some instances, it was difficult to determine whether a sample provided this negative readout because the amount of the nucleic acid present in the sample was too small to detect (was not sufficiently amplified) or because the gene product was not expressed; such data is referred to herein as “censored”. Genes identified as censored include insulin-like growth factor binding protein 1, which is a regulator of apoptosis; caspase 7, which encodes a protein that is stored in the mitochondrial intermembrane space and released into the cytosol after appropriate apoptotic stimuli, promoting apoptosis and interacting with calpain; and opioid growth factor receptor, which regulates cellular renewal and wound healing, and also inhibits pancreatic and squamous cell carcinomas. In addition, 3 ESTs and one nucleic acid sequence with a previously unknown function were also determined to be genes that were censored in polyps.

Genes with Increased Expression in Intestinal Polyps

A number of nucleic acid molecules were expressed in intestinal polyp samples but not in normal tissue. These nucleic acid molecules can be categorized into families based on function. For example, a number of genes involved in cell cycle or tumor suppression were identified, including CDK4/CDK6 inhibitor, which is a p19 regulator of passage through the G1 checkpoint of the cell cycle; RAN GTPase activating protein 1, which controls progression through the cell cycle by regulating the transport of proteins and nucleic acids across the nuclear membrane; prohibitin, which is a potential tumor suppressor, regulating E2F1 function; non-receptor protein tyrosine kinase Ack, which inhibits Ras-induced malignant phenotypes in fibroblasts; PRG1, which is an early-response gene transcriptionally induced by p53; and ubiquitin conjugating enzyme E2 variant 1, which is involved in the control of differentiation and the entry of a larger proportion of cells in the division cycle and an accumulation in G2-M of the cell cycle.

In addition, two genes involved in apoptosis were identified as exhibiting increased expression in intestinal polyps, TANK1, a tumor necrosis receptor-associated factor-(TRAF) interacting protein, which is a mediator of NFKB activation after induction by TRAF2, and an apoptosis inhibitor; and Epiregulin, an epidermal growth factor family member. Downregulation of the epidermal growth factor pathway leads to apoptosis.

Another family of genes identified as being upregulated in intestinal polyps is a group of genes involved in cell adhesion. For example, collagen type 1α, which directly interacts with laminin; E-cadherin, which is in direct contact with APC, a negative regulator of polyp formation; and Laminin β3, an upstream regulator of E-cadherin were identified as having increased expression in intestinal polyps.

In addition, a number of genes involved in transcription were identified as having increased expression in intestinal polyps, including Translation Initiation Factor EIF-2B-ε subunit; cleavage and polyadenylation specificity factor 73 kDa subunit; Nucleolin, which is involved in ribosome biogenesis, cytokinesis, nucleogenesis, cell proliferation and growth, and chromatin remodeling; cdc2/CDC28-like protein kinase 3 (Clk3), which includes one catalytically active and one inactive isoform, interacting with and inducing the nuclear redistribution of SR proteins; and hGAR1, which is a component of H/ACA snoRNPs and telomerase.

Other genes identified as having increased expression in intestinal polyps, compared to normal intestinal tissue include myosin IC, which is an unconventional crypt cell marker; carboxypeptidase E, which is a metallo carboxypeptidase family member that functions as a regulated secretory pathway sorting receptor, and is involved in the trimming of paired basic residues at the C terminus of prohormone-derived peptides. Other genes identified in the screen as being upregulated in intestinal polyps include cytochrome p450, which is involved in the metabolism of aromatic substances; thymidine kinase 1, which is involved in the pyrimidine salvage pathway, and is also a soluble, putative up-regulated c-Myc target gene; and Guanine-Binding Protein β-subunit, which is involved in GDP to GTP exchange.

In addition, a gene with similarities to the Glycogen phosphorylase gene, as well as 23 ESTs or sequences with previously unknown function were also identified in the above-described screen.

While this invention has been particularly shown and described with references to preferred embodiments thereof, it will be understood by those skilled in the art that various changes in form and details may be made therein without departing from the scope of the invention encompassed by the appended claims.

1-70. (canceled)

71. A method of identifying an intestinal polyp in a human comprising determining expression in a nucleic acid sample derived from intestinal tissue of the human of at least three nucleic acid molecules having increased expression in an intestinal polyp relative to a control, wherein increased expression of said nucleic acid molecules in said sample is indicative of an intestinal polyp.

72. The method according to claim 71 wherein the polyp is an upper intestinal polyp and the nucleic acid sample is derived from upper intestinal tissue.

73. The method according to claim 71, wherein the polyp is a colonic polyp and the nucleic acid sample is derived from colonic tissue.

74. The method according to claim 71, wherein the nucleic acid sample comprises DNA.

75. The method according to claim 71, wherein the nucleic acid sample comprises mRNA.

76. The method according to claim 71, wherein the step of determining expression is performed utilizing specific hybridization probes.

77. The method according to claim 76, wherein the hybridization probes are contained in an oligonucleotide microarray.

78. The method according to claim 71, wherein the step of determining expression is performed utilizing antibodies.

79. The method according to claim 71, wherein the nucleic acid molecules are selected from the group consisting of the nucleic acid molecules in FIGS. 1A-1U.

80. A method of identifying an intestinal polyp in a human comprising determining expression in a nucleic acid sample derived from intestinal tissue of the human of at least three nucleic acid molecules having decreased expression in an intestinal polyp relative to a control, wherein decreased expression of said nucleic acid molecules in said sample is indicative of an intestinal polyp.

81. The method according to claim 80 wherein the polyp is an upper intestinal polyp and the nucleic acid sample is derived from upper intestinal tissue.

82. The method according to claim 80, wherein the polyp is a colonic polyp and the nucleic acid sample is derived from colonic tissue.

83. The method according to claim 80, wherein the nucleic acid sample comprises DNA.

84. The method according to claim 80, wherein the nucleic acid sample comprises mRNA.

85. The method according to claim 80, wherein the step of determining expression is performed utilizing specific hybridization probes.

86. The method according to claim 85, wherein the hybridization probes are contained in an oligonucleotide microarray.

87. The method according to claim 80, wherein the step of determining expression is performed utilizing antibodies.

88. The method according to claim 80, wherein the nucleic acid molecules are selected from the group consisting of the nucleic acid molecules in FIGS. 1A-1U.

89. A method of identifying an intestinal polyp in a human comprising determining expression in a nucleic acid sample derived from intestinal tissue of the human of at least one nucleic acid molecule having increased expression in an intestinal polyp relative to a control, said nucleic acid molecule selected from the group consisting of the nucleic acid molecules in FIGS. 1A-1U, wherein increased expression of said nucleic acid molecules in said sample is indicative of an intestinal polyp.

90. A method of identifying an intestinal polyp in a human comprising determining expression in a nucleic acid sample derived from intestinal tissue of the human of at least one nucleic acid molecule having decreased expression in an intestinal polyp relative to a control, said nucleic acid molecule selected from the group consisting of the nucleic acid molecules in FIGS. 1A-1U, wherein decreased expression of said nucleic acid molecules in said sample is indicative of an intestinal polyp.
